(19) United States
(12) Patent Application Publication
(10) Pub. No.: US 2026/0274787 A1
(43) Pub. Date: Sep. 17, 2026

(54) SUSTAINABLE PREPARATION OF HEXAMETHYLENE DIISOCYANATE FOR THE PRODUCTION OF POLYURETHANE

(71) Applicant: Covestro Deutschland AG, Leverkusen (DE)

(72) Inventors: Andreas Bulan, Langenfeld (DE); Rainer Weber, Odenthal (DE)

(21) Appl. No.: 19/471,519

(22) PCT Filed: Apr. 4, 2024
(86) PCT No.: PCT/EP2024/059123

§ 371 Date: Oct. 1, 2025

(30) Foreign Application Priority Data
Apr. 6, 2023 (EP) 23166923.5

(51) Int. Cl. C07C 253/30; C01B 32/80; C07C 1/20; C07C 29/151; C07C 209/48; C07C 253/26; C08G 18/73; C25B 1/26

(52) U.S. Cl. CPC C07C 253/30; C01B 32/80; C07C 1/20; C07C 29/1518; C07C 209/48; C07C 253/26; C08G 18/73; C25B 1/26

(57) ABSTRACT

A method of preparing hexamethylene diisocyanate and of preparing polyurethane from hexamethylene diisocyanate is provided. Methanol is used in the method as a raw material for the build-up of the hexamethylene moiety of the hexamethylene diisocyanate.

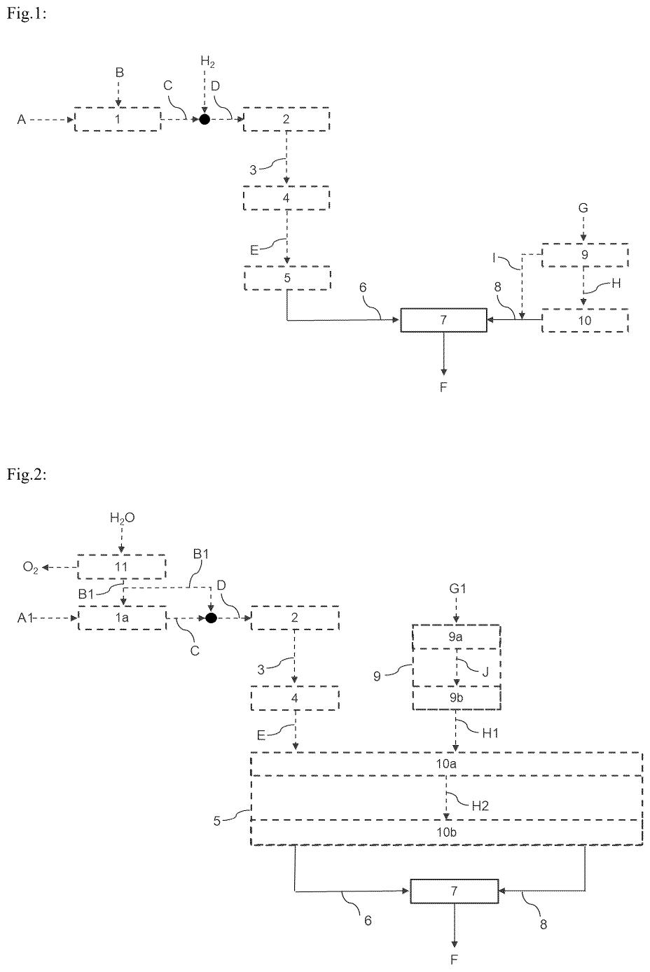

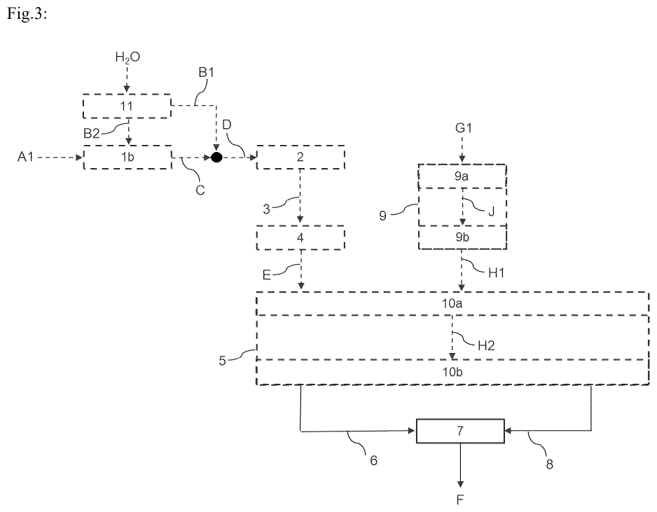

### CROSS REFERENCE TO RELATED APPLICATIONS

This application is a U.S. national stage application, filed under 35 U.S.C. § 371, of International Application No. PCT/EP2024/059123, which was filed on Apr. 4, 2024, and which claims priority to European Patent Application No. 23166923.5, which was filed on Apr. 6, 2023. The entire contents of each are hereby incorporated by reference into this specification.

### FIELD

The present invention relates to a production process of hexamethylene diisocyanate for the synthesis of polyurethane, to a process for producing polyurethane from the hexamethylene diisocyanate obtained according to the aforementioned production process and to a multicomponent system for hexamethylene diisocyanate production according to the aforementioned production process.

### BACKGROUND

Industrial production of hexamethylene diisocyanate (also known as HDI, as hexane-1,6-diyl diisocyanate or as 1,6-diisocyanatohexane) has hitherto been based predominantly on the use of fossil raw materials, for example synthesis gas provided from natural gas and petroleum-based aromatic compounds. The systematic use of renewable raw materials and/or of byproducts from wastes for the production of materials such as hexamethylene diisocyanate is an objective for the provision of sustainable plastics, such as polyurethane and materials derived therefrom.

The utilization of energy from renewable energy sources for industrial production of chemical raw materials, for example of hexamethylene diisocyanate, is also desirable.

### SUMMARY

It was accordingly an object of the present invention to provide a more sustainable production process for hexamethylene diisocyanate on an industrial scale which makes it possible to utilize renewable raw materials. It was also a further object of the invention to provide a method for producing hexamethylene diisocyanate which, despite possible variations in the availability of renewable energy, makes it possible to achieve a low-emission hexamethylene diisocyanate for production of polyurethanes. “Renewable energy” is understood by those skilled in the art to mean energy from an inexhaustible energy source, for example wind power, hydro power, bioenergy (e.g. conversion of biogas or biomass into electricity) or solar power.

The “sustainability” of a process is understood by those skilled in the art according to the definition of sustainability (sustainable development) coined by the UN in the Brundtland Report of the “World Commission on Environment and Development”, this being that the execution of the process in the present makes the smallest possible contribution, or none at all, to compromising the ability of future generations to meet their own needs, in particular needs in respect of the use of resources such as fossil raw materials and especially in respect of the conservation of living space, for example the protection of the earth's atmosphere. It is accordingly an object of the invention to make the production of hexamethylene diisocyanate and polyurethane produced therefrom more sustainable than the production methods known from the prior art. The contribution of the production of hexamethylene diisocyanate and of polyurethane to decreasing satisfaction of the needs of future generations should be reduced or avoided.

A first subject of the invention is thus a process for producing hexamethylene diisocyanate for the production of polyurethane comprising at least the steps of:
a) provision of hexamethylenediamine which is a process product of a process comprising at least the following steps:
   i) provision of methanol which is a process product of a process comprising at least the following steps:
      i-1) provision of carbon monoxide as a process product of at least a partial reduction of CO₂ to CO and/or as a process product of at least a partial oxidation of organic material to CO, wherein at least one organic material is selected from at least one organic, solid compound, methane from a biological source or mixtures thereof;
      i-2) conversion of the provided carbon monoxide into methanol;
   ii) synthesis of propene from previously provided methanol by at least the following steps:
      ii-1) conversion of the previously provided methanol to afford a product mixture containing dimethyl ether, water and methanol;
      ii-2) conversion of starting material containing dimethyl ether, water and methanol in each case from the aforementioned product mixture at a temperature of more than 200° C. by contact with a catalyst, preferably with at least one zeolite compound as catalyst, into propene;
   (iii) synthesis of acrylonitrile from the propene by at least the following steps:
      iii-1) provision of ammonia which is a process product of a process comprising at least the following steps:
         iii-1-1) provision of hydrogen gas as a product of an electrolysis of water, preferably using electrical energy generated from renewable energy;
         iii-1-2) reaction of the provided hydrogen gas with gaseous nitrogen to afford ammonia;
      iii-2) reaction of the propene with the provided ammonia to afford acrylonitrile;
   iv) synthesis of adiponitrile from the acrylonitrile by at least the following steps:
      introduction of a mixture containing water, said acrylonitrile and at least one electrolyte salt into the cathode space of an electrolysis cell and contacting the mixture with a cathode having an electrical current applied to it;
      cathodic hydrodimerization of acrylonitrile to form an adiponitrile-containing product mixture;
      discharging of the adiponitrile-containing product mixture from the cathode space of the electrolysis cell and optional purification of the adiponitrile;
   v) synthesis of hexamethylenediamine from the adiponitrile by at least the following steps:
      provision of hydrogen gas as a product of an electrolysis, preferably using electrical energy generated from renewable energy;
      hydrogenation of the adiponitrile with the hydrogen gas to afford hexamethylenediamine;
b) production of phosgene by at least the following process steps:
   i) provision of carbon monoxide as a process product of at least a partial reduction of CO₂ to CO and/or a process product of at least a partial oxidation of organic material to CO, wherein at least one organic material is selected from at least one organic, solid compound, an organic, liquid monohydroxyalkyl compound, methane from a biological source or mixtures thereof;
   ii) reaction of the carbon monoxide from step i) with chlorine to afford phosgene;
c) reaction of the provided hexamethylenediamine with the phosgene to afford hexamethylene diisocyanate.

### DETAILED DESCRIPTION

According to the invention, a “catalyzed reaction” or “catalytic reaction” is carried out using a catalyst which catalyzes the product formation from at least one reactant (for example carbon dioxide or methanol) compared to the same reaction under the same reaction conditions but in the absence of the catalyst by reducing the energy required and/or by enhancing selectivity to increase product yield. For example, the conversion according to step a) ii-2) is a catalytic reaction of said starting material to afford propene.

According to the present invention a material or a chemical compound is organic when the material/the chemical compound contains at least one covalent carbon-hydrogen bond.

A substance/a material is defined as solid when the substance is in the form of a solid at 25° C. and 1013 mbar. A substance/a material is defined as liquid when the substance is in the form of a liquid at 25° C. and 1013 mbar.

In step a) of the process according to the invention hexamethylenediamine (also known as 1,6-diaminohexane or as hexane-1,6-diamine) is provided. For provision of the hexamethylenediamine it is sufficient according to the invention when the provided hexamethylenediamine is an actual process product of at least the steps i), ii), iii), iv) and v) recited under a). This means that it is sufficient for performing the step of provision of the hexamethylenediamine merely to withdraw the hexamethylenediamine produced in the recited manner from a storage vessel or from a feed conduit in the context of a delivery as raw material in order to supply it at least to step c) of the process according to the invention. In this case, the hexamethylenediamine producer, as the performer of the process according to the invention, does not themself perform the steps i) and ii) for producing the hexamethylenediamine recited under a) of the process according to the invention but rather only ensures that the provided hexamethylenediamine was produced by application of at least the steps i) and ii) recited under a) and is thus the actual process product thereof.

According to the invention it is likewise possible that for provision of the hexamethylenediamine at least the steps i) to v) for producing the hexamethylene diamine recited under a) are performed by the hexamethylene diisocyanate producer as integral steps of a process according to the invention for producing hexamethylene diisocyanate and hexamethylenediamine obtained thereby, optionally after intermediate storage in a storage vessel, is supplied at least to step c) of the process according to the invention.

The possibilities for provision, i.e. supply or in-house production of the specific hexamethylenediamine by the hexamethylene diisocyanate producer, in each case also apply to the following embodiments of the steps i) to v) recited under a).

Step i) recited under a) in the process according to the invention requires the provision of methanol which is an actual process product of a process comprising at least the following steps:
i-1) conversion of CO into methanol, wherein the CO is a process product of at least a partial reduction of CO₂ to CO and/or a process product of at least a partial oxidation of organic material to CO, wherein at least one organic material is selected from at least one organic, solid compound, methane from a biological source or mixtures thereof.

According to the present invention it is sufficient for the provision of the methanol if the methanol is an actual process product of at least the reaction recited under i-1), wherein this reaction utilizes carbon monoxide (carbon monoxide also referred to as CO) which is in turn an actual process product of a specific process. This means that it is sufficient for performing the step of provision of the methanol to merely withdraw the methanol produced in the recited manner from a storage vessel or from a feed conduit in the context of a delivery as raw material. In this case, the hexamethylene diisocyanate producer, as the performer of the process according to the invention, or the supplier of the hexamethylenediamine provided for the process according to the invention does not themself perform the aforementioned reaction to produce the methanol but rather only ensures that the methanol provided for providing the hexamethylenediamine was produced by application of at least said step i-1) and is thus the actual process product thereof.

It is also possible according to the invention that, for provision of the methanol, at least the aforementioned conversion to produce the methanol is performed by the hexamethylene diisocyanate producer or the supplier of the provided hexamethylenediamine as an integral step of the process according to the invention for producing hexamethylene diisocyanate and the methanol obtained thereby, optionally after intermediate storage in a storage vessel, is supplied to the synthesis of hexamethylenediamine provided for under step a) of the process while performing at least the steps ii-1), ii-2), iii), iv) and v).

The possibilities for provision, i.e. supply or in-house production of the specific methanol by the hexamethylene diisocyanate producer, in each case also apply to the following embodiments of the provision of the methanol.

The production of methanol typically comprises reacting carbon oxides with hydrogen (the mixture also referred to as synthesis gas). This process is exothermic and can be described by the following reaction equations:

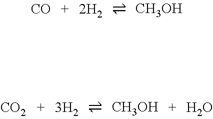

Both reactions are coupled by the likewise exothermic water gas shift reaction which can be described as follows:

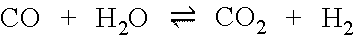

Conventionally, methanol synthesis employs synthesis gas mainly composed of CO and H₂ which is in turn produced from natural gas (fossil methane) and steam in a reformer process (steam reforming).

The composition of the synthesis gas has a great influence on optimal utilization. The composition may be described by the stoichiometry number (SN):

$$S N = \frac{{n}_{{H}_{2}} - {n}_{{CO}_{2}}}{{n}_{CO} + {n}_{{CO}_{2}}}$$

When SN=2, the reactants are present in a stoichiometric ratio according to the aforementioned reaction equations. In reality, however, slightly higher values for SN (2.01-2.1) are used, which is achieved by a higher hydrogen content in the synthesis gas (Dittmeyer et al. “Chemische Technik” [Chemical Technology], volume 4, 5th edition).

In the process according to the invention, the carbon monoxide required for the conversion for methanol provision is not provided by conventional steam reforming using fossil carbon sources (natural gas) but rather by partial reduction of carbon dioxide and/or by partial oxidation (and if necessary gasification) of said organic material to CO.

In the process according to the invention the partial oxidation of organic solid material (preferably of organic, polymeric material) comprises provision of CO from organic solid material (preferably from used polymer waste fractions) and oxygen-containing gas by conversion of said organic solid material by partial oxidation to a gas containing CO (hereinbelow also referred to as gasification).

The use of polymer-containing waste fractions, which may be composed predominantly of PET-, PE-, PP-, polyurethane- or polycarbonate-containing waste fractions for example (hereinbelow referred to as polymer waste fraction), for provision of CO means that these polymer-containing waste fractions are supplied for recycling, thus producing hexamethylene diisocyanate and also polyurethane from this hexamethylene diisocyanate with improved sustainability.

A “polymeric compound” is a molecule having a relative molecular mass (Mw) of at least 2000 g/mol, the chemical structure of which comprises mostly multiply repeating structural units derived from one or more different molecules of lower relative molecular mass. The average molar masses specified in the scope of this application for polymers or polymeric compounds are—unless explicitly otherwise stated—always weight-average molar masses Mw, which can in principle be determined by gel-permeation chromatography using an RI detector, it being expedient to perform the measurement against an external standard. A “polymeric material” is a material containing at least one polymeric compound.

The partial oxidation causes organic material (preferably organic solid material, particularly preferably polymeric organic material, very particularly preferably the polymer waste faction) and oxygen to undergo a partial oxidation reaction to afford a product gas mixture containing hydrogen and CO and optionally byproducts. Byproducts are, in particular, hydrocarbons having 1 to 8 carbon atoms. Also present in the product gas mixture are CO₂ and water vapor.

A further byproduct obtained in the gasification process is a residual fraction that cannot be reacted further.

The temperature required for gasification is at least partially achieved by partial combustion (partial oxidation) of the organic solid material (preferably of the polymeric organic material, particularly preferably of the polymer waste fraction) with an oxygen-containing gas. The partial oxidation of the organic solid material introduced is performed in the reactor at a temperature of at least 400° C.

Preferably, the partial oxidation of the material in the reactor is carried out at a temperature in a temperature range of from 600° C. to 1500° C., especially from 850° C. to 1400° C., more preferably from 1100° C. to 1300° C.

The carbon monoxide provided as a process product of a partial oxidation may for example have been produced by a process for producing carbon monoxide for the provision of methanol containing at least the following steps:
1) provision of an oxygen-containing gas stream containing at least 50% by weight of oxygen gas,
2) partial oxidation of organic material (preferably organic material containing at least one polymeric organic compound), wherein said organic material is introduced into a reactor of an apparatus and is therein treated at least by supplying said oxygen-containing gas stream and heat at at least 400° C. to form a product gas and the resulting product gas, optionally after at least one further partial oxidation step of the product gas, is discharged from the apparatus as a carbon monoxide-containing product gas stream together with particulate solid dispersed therein;
3) supply of the carbon monoxide-containing product gas stream to a purification in which at least
   3-1) the carbon monoxide-containing product gas stream is supplied to a scrubbing step for separation of solid, wherein
      (i) water is brought into contact with the carbon monoxide-containing product gas stream, as a result of which the particulate solid dispersed in the carbon monoxide-containing product gas stream forms a slag and this slag is removed and discharged,
      (ii) the carbon monoxide-containing product gas stream purified of particulate solid is discharged;
   3-2) a carbon monoxide-containing product gas stream purified of particulate solid by a scrubbing step is in a drying step supplied to at least one water separation, water is separated and the resulting carbon monoxide-containing product gas stream is discharged,
   3-3) a carbon monoxide-containing product gas stream purified by separation of water is supplied to at least one carbon dioxide separation, carbon dioxide is separated and the resulting carbon monoxide-containing product gas stream and carbon dioxide are discharged;
   3-4) a carbon monoxide-containing product gas stream purified by separation of carbon dioxide is supplied to at least one separation unit for separating carbon monoxide, a separation of carbon monoxide is performed and the resulting carbon monoxide and a hydrogen gas-containing residual gas is discharged;
   3-5) optionally a hydrogen gas-containing residual gas separated using said separation unit is supplied to a residual gas treatment, hydrogen gas is separated and hydrogen gas and an end gas are discharged.

A solid is known to be “particulate” when it is in the form of a granular mixture of a multitude of loose, solid particles of said substance, which in turn comprises what are known as grains. A grain is a term for the particulate constituents of powders (grains are the loose, solid particles), dusts (grains are the loose, solid particles), granules (loose, solid particles are agglomerates of a plurality of grains), and other granular mixtures.

A “reactor” is a volume in which a chemical transformation, for example a partial oxidation of a polymeric organic compound of a material, takes place. For the partial oxidation, this can for example be the volume of a heated vessel in which the material is contained.

The oxygen gas required for the partial oxidation may be withdrawn from a water electrolysis or an air separation plant for example. In a preferred embodiment of the partial oxidation, the oxygen-containing gas stream is provided by performing an electrolysis of water to obtain oxygen gas and hydrogen gas and the oxygen gas from this electrolysis is utilized to provide the oxygen-containing gas stream.

A water electrolysis may be performed with prior art plants. Industrial systems for alkaline water electrolysis as well as for polymer electrolyte-based electrolysis, so-called PEM electrolysis, are known and commercially available. The principles of water electrolysis are described by way of example in chapter 6.3.4 in Volkmar M. Schmidt in “Elektrochemische Verfahrenstechnik” [Electrochemical process technology](2003 Wiley-VCH-Verlag; ISBN 3-527-29958-0).

According to the invention, the organic solid material introduced into the reactor for the partial oxidation preferably contains at least one polymeric organic compound. The partial oxidation should take place in the reactor as uniformly and selectively as possible. An increase in these parameters can be achieved when in a preferred embodiment, in each case based on the time prior to introduction, the weight ratio of the oxygen gas present in the oxygen-containing gas stream to the polymeric organic compound is within a weight ratio range from 0.4:1.0 to 1.2:1.0, preferably from 0.6:1.0 to 0.9:1.0.

In a preferred embodiment, the partial oxidation of the introduced organic solid material is performed at an absolute pressure of more than 1 bar, preferably at an absolute pressure in a range from 2 to 80 bar, particularly preferably at an absolute pressure in a range from 2 to 50 bar.

The partial oxidation of the organic solid material may be carried out in at least one of the following three reactors: reactor for entrained-flow gasification, reactor for fluidized-bed gasification, and reactor for fixed-bed gasification.

For use in a reactor for entrained-flow gasification, the organic solid material must be ground to a particle size having a median particle diameter X_{50.3} of <0.1 mm (dust). The supply to the reactor is effected either pneumatically or as a slurry. The greatest limitation of this type of partial oxidation for the chemical recycling of waste is the grindability and conveyability of starting materials from heterogeneous waste. A thermal treatment of biomass (torrefaction) at 200-300° C. with exclusion of O₂ is used to produce a “biochar” with similar grindability to coal. In another variant, an upstream pyrolysis can be used to produce a pumpable pyrolysis oil. The oil obtained by waste pyrolysis can be partially oxidized either directly or in the form of a slurry mixed with the solid pyrolysis residue (pyrolysis coke). This process configuration was developed by Noell (Noell-Konversionsverfahren zur Verwertung und Entsorgung von Abfällen [Noell conversion process for the recovery and disposal of waste], Jürgen Carl EF-Verl. für Energie- und Umwelttechnik, 1994, ISBN: 3924511829).

Another variant is the use of a reactor for fluidized-bed gasification. The gasification of organic solid material, especially of waste, in fluidized-bed reactors, is well known through the technologies of EBARA (Showa Denko, Japan) and ENERKEM (Enerkem, Edmonton, Canada) and the large-scale demonstration of high-temperature Winkler gas production (HTW) by Rheinbraun AG (now RWE) from 1993 to 1997 in Berrenrath, Germany. The pretreatment for introducing the organic material or the material containing polymeric organic compounds into the reactor requires a comminution to a median particle diameter X_{50.3} of 30-80 mm. Introduction into the reactor vessel is effected via screw conveyors and limits the gasifier pressure to not more than 10 bar. The ENERKEM and EBARA technologies permit the gasification of high-caloric waste (plastic waste or plastic-rich substitute fuels). Fluidized-bed gasifiers are operated at mild temperatures of 700-950° C., well below the ash melting point of the feedstock, in order to avoid caking and agglomeration in the reactor. A further advantage of this mild reactor temperature is the incomplete carbon turnover in the fluidized bed. Furthermore, the crude gas from reactors for fluidized-bed gasifiers typically contains significant amounts of methane and other hydrocarbons. To compensate for this and to ensure a high syngas yield of H₂ and CO, the ENERKEM and EBARA processes employ a second high-temperature stage for partial oxidation (approx. 1400° C.) arranged directly downstream of the fluidized bed, in order to melt fly ash and convert hydrocarbons in the product gas from the first stage into the final carbon monoxide-containing product gas stream. ENERKEM calls this second stage “thermoreformer”, while EBARA refers to a “high-temperature gasification furnace”. The high temperature in this second partial oxidation stage increases CO₂ production.

The CO for the provision of the methanol may likewise preferably be a process product derived from the conversion of methane from a biological source by partial oxidation in a classical reformer process. In the context of a preferred embodiment of such a provision, the CO is a process product of a reaction of at least methane from a biological source and steam (particularly preferably with addition of CO₂) with supply of thermal energy at a temperature of at least 500° C. to afford carbon monoxide. A further suitable sustainable reformer process with addition of CO₂ is described in the PCT patent application having application number PCT/EP2022/052267, the contents of which are hereby fully and expressly incorporated by reference. This process relates to the production of carbon monoxide from methane from a biological source, steam and CO₂ comprising at least the steps of:
synthesis of carbon monoxide in a reformer process in which methane from a biological source and steam are reacted with addition of at least CO₂ and with supply of thermal energy at a temperature of at least 500° C. to afford carbon monoxide-containing product gas, purification of the carbon monoxide-containing product gas obtained from the aforementioned
synthesis, at least by separation of CO₂ and optionally also by at least one separation selected from separation of water, separation of hydrogen or a combination thereof, to obtain carbon monoxide;
provision of CO₂ for said addition to the aforementioned reformer process at least from said separation of CO₂ in the aforementioned purification step.

It is in turn preferable when the thermal energy supplied to the reformer process for synthesis of carbon monoxide is provided by at least one method selected from (i) combustion of fuel containing hydrogen produced by means of renewable energy, (ii) combustion of fuel containing methane from a biological source, (iii) conversion of electrical energy generated from renewable energy into heat.

“Methane from a biological source” (also referred to as “biomethane”) is considered by a person skilled in the art to mean methane obtained industrially from biomass by methane fermentation as opposed to fossil methane. Methane fermentation is known to mean the anaerobic degradation of organic substances by microorganisms. Methane from a biological source is produced, for example, in biogas plants in which both organic wastes and renewable raw materials are correspondingly fermented.

“Renewable energy” is understood by those skilled in the art to mean energy from an inexhaustible energy source, for example wind energy, hydro energy, bioenergy (e.g. conversion of biogas or biomass to power) or solar energy. Suitable renewable energy is therefore most preferably either wind power, solar energy, hydro power or mixtures thereof.

It is preferable according to the invention when the methanol provided according to i) of process step a) according to the invention is a process product of a reaction of CO, wherein this CO employed in the reaction is a process product of at least a partial reduction of CO₂ to CO, especially selected from a reverse water gas shift reaction using H₂ produced by electrolysis (preferably by water electrolysis, i.e. electrolysis of water), from an electrochemical reduction of CO₂ to CO or from mixtures thereof.

In a particularly preferred embodiment of the process according to the invention the provided methanol is provided as a process product of a conversion of CO, wherein the CO employed in the conversion is a process product produced by at least a partial production of a gas stream containing at least COₓ where x=1 or 2 and optionally hydrogen gas.

It has further proven preferable when in one embodiment of the process methanol is obtained by conversion of CO₂ with or without hydrogen and is utilized for the provision of methanol. It is particularly preferred in turn when the reaction is an electrochemical reaction, a homogeneously catalyzed reaction or a heterogeneously catalyzed reaction. In a further embodiment the CO₂ used therefor derives from a further CO₂ source, for example CO₂ emitted in the provision of thermal energy or CO₂ from an external CO₂ source, for example an industrial offgas.

According to the invention, an “electrochemical reaction” is carried out by application of electrical current in the reaction medium (e.g. via at least one electrode immersed into the reaction medium) in the presence of at least one reactant (e.g. carbon dioxide).

A “catalyzed reaction” or “catalytic reaction” is as defined hereinabove (vide supra). According to the invention, a “homogeneously catalyzed reaction” is carried out using a homogeneous catalyst and a “heterogeneously catalyzed reaction” using a heterogeneous catalyst.

A preferred CO₂ source is at least one “external CO₂ source” which contributes CO₂ that is not emitted by the process according to the invention. An external CO₂ source would be for example the CO₂ generated in cement production, in H₂ production for ammonia synthesis, in a fermentation, in offgas from combustion of fuels (for example waste incineration) or CO₂ obtained from the air.

In a particularly preferred embodiment of the process according to the invention, the carbon monoxide for methanol synthesis is produced from CO₂ in a reverse water-gas shift (RWGS) reaction zone through at least a reaction of hydrogen and CO₂ to afford carbon monoxide. Such an embodiment of carbon monoxide production is described for example in WO 2021/089737 A, the contents of which are fully and expressly incorporated herein by reference.

A “reaction zone” is the part of a reaction space in which a chemical reaction, for example the reverse water gas shift reaction, proceeds. A “reaction space” is a volume in which the coreactants taking part in a chemical reaction are brought together and in which the chemical reaction takes place. For a chemical reaction, this may, for example, be the volume of a vessel in which a reactant, for example carbon dioxide in the case of an RWGS reaction, and the coreactant thereof, hydrogen in the case of an RWGS reaction, are present together and are reacted in the reaction zone. This volume may be in a reactor for example.

The hydrogen gas utilized for the RWGS reaction is preferably provided by electrolysis, especially by chloralkali electrolysis or water electrolysis, particularly preferably by water electrolysis. This in turn preferably in each case uses electrical energy produced from renewable energy (especially from hydropower, solar power or wind power).

In a particularly preferred process, the carbon monoxide provided for provision of the methanol is a product of a process comprising at least the following steps:
provision of a CO₂ gas stream,
purification of the CO₂ gas stream from secondary constituents, in particular nitrogen oxides, sulfur compounds, dust, water, oxygen and HCl, optionally by adsorption, gas scrubbing or catalytic treatment to obtain a purified carbon dioxide,
introduction of hydrogen gas provided together with the purified CO₂ gas stream into an RWGS reaction zone and reaction of the reactants according to the principle of the RWGS reaction to afford a product gas mixture composed of steam, CO and optionally by-products, especially lower hydrocarbons, especially preferably methane,
separation of unreacted carbon dioxide from the gas mixture of the RWGS reaction obtained from the separation, in particular by amine scrubbing, and recycling the unreacted carbon dioxide to the RWGS reaction,
separation of the hydrogen unconverted in the RWGS reaction from the gas mixture of carbon monoxide and hydrogen obtained after the separation, in particular using a cold box, and optionally recycling the hydrogen into the RWGS reaction,
discharging of the remaining carbon monoxide from the separation.

The CO₂ source for provision of the CO₂ gas stream is for example the CO₂ emitted during provision of thermal energy for the process according to the invention (e.g. for the RWGS reaction), the CO₂ obtained from an air separation or CO₂ from a further external CO₂ source.

An external CO₂ source contributes CO₂ which is not emitted by the process according to the invention or embodiments thereof. An external CO₂ source would be, for example, the CO₂ which is formed in the offgas in cement production or on combustion of fuels (e.g. waste incineration), or CO₂ obtained from the air. This CO₂ from an external CO₂ source, in a preferred embodiment of the process of the invention, will be effected by absorption of a CO₂ fraction from (i) process gases or off gases selected from at least one process selected from cement production, H₂ production, incineration, and/or (ii) from air by introduction into alkali metal hydroxide solution, for example potassium hydroxide solution. This results in the formation of potassium hydrogencarbonate, which can then be thermally decomposed back to CO₂ and potassium hydroxide. The CO₂ released is then fed to the process of the invention for synthesis of carbon monoxide.

The RWGS reaction is preferably performed in the reaction zone at a temperature≥650° C., particularly preferably ≥700° C., very particularly preferably ≥750° C.

The RWGS reaction is preferably conducted in the presence of at least one catalyst. The latter is more preferably selected from at least one compound from the group of:
(I) mixed metal oxides of the formula A_{(1-w-x)}A′_{w}A″ₓB_{(1-y-z)}B′_{y}B″_{z}O₃-delta
   where:
   A, A′ and A″ are independently selected from the group of: Mg, Ca, Sr, Ba, Li, Na, K, Rb, Cs, Sn, Sc, Y, La, Ce, Pr, Nd, Pm, Sm, Eu, Gd, Tb, Dy, Ho, Er, Tm, Yb, Tl, Lu, Ni, Co, Pb, Bi and/or Cd; and
   B, B′ and B″ are independently selected from the group of: Cr, Mn, Fe, Bi, Cd, Co, Cu, Ni, Sn, Al, Ga, Sc, Ti, V, Nb, Ta, Mo, Pb, Hf, Zr, Tb, W, Gd, Yb, Mg, Li, Na, K, Ce and/or Zn; and

$$0 \leq w \leq 0.5 ; 0 \leq x < 0.5 ; 0 \leq y \leq 0.5 ; 0 \leq z \leq 0.5 and - 1 \leq delta \leq I ;$$

(II) mixed metal oxides of the formula A_{(1-w-x)}A′_{w}A″ₓB_{(1-y-z)}B′_{y}B″_{z}O_{3-delta} where:
   A, A′ and A″ are independently selected from the group of: Mg, Ca, Sr, Ba, Li, Na, K, Rb, Cs, Sn, Sc, Y, La, Ce, Pr, Nd, Sm, Eu, Gd, Th, Dy, Ho, Er, Tm, Yb, Tl, Lu, Ni, Co, Pb and/or Cd; and
   B is selected from the group of: Cr, Mn, Fe, Bi, Cd, Co, Cu, Ni, Sn, Al, Ga, Sc, Ti, V, Nb, Ta, Mo, Pb, Hf, Zr, Th, W, Gd, Yb, Mg, Cd, Zn, Re, Ru, Rh, Pd, Os, Ir and/or Pt; and
   B′ is selected from the group of: Re, Ru, Rh, Pd, Os, Ir and/or Pt; and B″ is selected from the group of: Cr, Mn, Fe, Bi, Cd, Co, Cu, Ni, Sn, Al, Ga, Sc, Ti, V, Nb, Ta, Mo, Pb, Hf, Zr, Th, W, Gd, Yb, Mg, Cd and/or Zn; and

$$0 \leq w \leq 0.5 ; 0 \leq x \leq 0.5 ; 0 \leq y \leq 0.5 ; 0 \leq z \leq 0.5 and - I \leq delta \leq 1 ;$$

(III) mixtures of at least two different metals M1 and M2 on a support comprising an oxide of Al, Ce and/or Zr that has been doped with a metal M3;
   where: M1 and M2 are independently selected from the group of: Re, Ru, Rh, Ir, Os, Pd and/or Pt; and
   M3 is selected from the group of: Sc, Y, La, Ce, Pr, Nd, Sm, Eu, Gd, Th, Dy, Ho, Er, Tm, Yb and/or Lu;
(IV) mixed metal oxides of the formula LOₓ(M_{(y/z)}Al_{(2-y/z)}O₃)_{z}; where:
   L is selected from the group of: Na, K, Rb, Cs, Mg, Ca, Sr, Ba, Sc, Y, Sn, Pb, Pd, Mn, In, Tl, La, Ce, Pr, Nd, Sm, Eu, Gd, Tb, Dy, Ho, Er, Tm, Yb and/or Lu; and
   M is selected from the group of: Ti, Zr, Hf, V, Nb, Ta, Cr, Mo, W, Mn, Re, Fe, Ru, Os, Co, Rh, Ir, Ni, Pd, Pt, Zn, Cu, Ag and/or Au; and

$$1 < x \leq 2 ; 0 < y \leq 12 ; and 4 \leq z \leq 9 ;$$

(V) mixed metal oxides of the formula LO(Al₂O₃)_{z}; where:
   L is selected from the group of: Na, K, Rb, Cs, Mg, Ca, Sr, Ba, Sc, Y, Sn, Pb, Mn, In, Tl, La, Ce, Pr, Nd, Sm, Eu, Gd, Tb, Dy, Ho, Er, Tm, Yb and/or Lu; and

$$4 \leq z \leq 9 ;$$

(VI) oxidic catalyst comprising Ni and Ru;
(VII) metal M1 and/or at least two different metals M1 and M2 on and/or in a support, where the support
   is a carbide, oxycarbide, carbonitride, nitride, boride, silicide, germanide and/or selenide of metals A and/or B; where:
   M1 and M2 are independently selected from the group of: Cr, Mn, Fe, Co, Ni, Re, Ru, Rh, Ir, Os, Pd, Pt, Zn, Cu, La, Ce, Pr, Nd, Sm, Eu, Gd, Tb, Dy, Ho, Er, Tm, Yb and/or Lu; and
   A and B are independently selected from the group of: Be, Mg, Ca, Sc, Ii, V, Cr, Mn, Fe, Co, Ni, Y, Zr, Nb, Mo, Hf, Ta, W, La, Ce, Pr, Nd, Sm, Eu, Gd, Tb, Dy, Ho, Er, Tm, Yb and/or Lu;
and/or
reaction products of (I), (II), (III), (IV), (V), (VI) and/or (VII) in the presence of carbon dioxide, hydrogen, carbon monoxide and/or water at a temperature of ≥700° C.

In an equally preferred embodiment of the process the methanol provided for the provision of the hexamethylenediamine in the process according to the invention is provided from carbon monoxide, wherein the carbon monoxide is a product of an electrochemical partial reduction of carbon dioxide to afford carbon monoxide. In this embodiment, CO provision is effected by introducing a CO₂ gas stream into an electrolysis apparatus and reducing said stream to carbon monoxide at an electrode, preferably at a gas diffusion electrode (particularly preferably using electrical energy produced from renewable energy). This electrochemical partial reduction is hereinbelow also referred to as CO₂ electrolysis.

The CO₂ electrolysis may be, for example, a high-temperature electrolysis which is operated at a temperature of more than 600° C., possibly with the addition of water for production of synthesis gas.

High-temperature electrolyses are known in principle and available on the market, e.g. from Haldor Topsoe, eCOs®. High-temperature electrolysis forms oxygen at the anode.

If the CO₂ electrolysis is operated as a low-temperature electrolysis, the electrolysis takes place at a temperature below 150° C.

With all CO₂ electrolyses, the CO₂ gas is supplied to the cathode space.

In the case of low-temperature electrolysis, CO₂ is in particular converted to carbon monoxide and possibly hydrogen at a gas diffusion electrode. The person skilled in the art is aware, for example from WO 2021/069470 A, of electrodes and a method for performing an electrochemical reduction of CO₂. The electrochemical reduction of CO₂ is preferably performed by a process of WO 2021/069470 A. This document is fully and expressly incorporated herein by reference. In this preferred electrolytic process for production of carbon monoxide, carbon monoxide, optionally hydrogen and chlorine are obtained by electrochemical conversion of carbon dioxide and alkali metal chloride solution. This preferred electrolysis process is characterized in that the carbon dioxide is electrochemically reduced at a gas diffusion electrode as cathode in an aqueous alkali metal chloride-containing solution as catholyte and, at the same time, chlorine is produced anodically from an aqueous alkali metal chloride-containing solution as anolyte, where the alkali metal salt of the carbonic acid formed in the catholyte, selected from alkali metal carbonate, alkali metal hydrogencarbonate or mixtures thereof, is then reacted with hydrogen chloride to give carbon dioxide and alkali metal chloride, and the carbon dioxide released is returned to the cathode space for the gas diffusion electrode and the alkali metal chloride produced is returned either to the anode space and/or to the cathode space.

According to the known principles, an MEA (membrane-electrode assembly) concept can also be used in low-temperature electrolysis. In this case, a catalyst is applied to the membrane. An upstream gas diffusion layer regulates gas and liquid transport. This can be effected on both the anode and the cathode side. It is also possible to bring a gas diffusion electrode into direct contact with the membrane.

The gas diffusion electrode used may be installed in the electrolysis cell in a zero-gap or else in a finite-gap arrangement. A preferred arrangement for a low-temperature electrolysis of CO₂ is described in document WO 2020/057998 A1, which is fully and expressly incorporated by reference.

The cathode space, or the gas diffusion electrode installed therein, can be supplied with an excess of CO₂. “Excess” means introducing more CO₂ than would be necessary for the stoichiometric conversion on the basis of the flowing electric current. A gas mixture consisting of unreacted CO₂, CO and H₂ thus leaves the cathode space.

In a further embodiment of CO₂ electrolysis it is preferable when the electrical energy used therefor is electrical energy produced from renewable energy, especially electrical energy produced from wind power, solar power or hydropower.

If the carbon monoxide utilized for providing the methanol is a process product of at least one of the aforementioned processes, the reaction of the CO to afford methanol may employ at least one of the processes known therefor to those skilled in the art, proceeding from CO-containing synthesis gas.

If step ii-1) does not in any event directly afford CO in admixture with sufficient hydrogen as synthesis gas, in case of a lack of a sufficient amount of hydrogen gas the CO is admixed with hydrogen gas to afford synthesis gas before the conversion into methanol. This hydrogen gas additionally added to the CO in the context of a provision of methanol is preferably provided by electrolysis, especially by chloralkali electrolysis or water electrolysis. Water electrolysis can be carried out with prior art plants. Industrial systems for alkaline water electrolysis as well as for polymer electrolyte-based electrolysis, so-called PEM electrolysis, are known and commercially available. The principles of water electrolysis are described by way of example in chapter 6.3.4 in Volkmar M. Schmidt in “Elektrochemische Verfahrenstechnik” [Electrochemical process technology](2003 Wiley-VCH-Verlag; ISBN 3-527-29958-0). In a further embodiment, it is preferable when the electrical energy used for the electrolysis for hydrogen production is electrical energy produced from renewable energy, especially electrical energy produced from wind power, solar power or hydropower.

Particular preference is given to an embodiment in which the methanol to be provided for the process according to the invention has correspondingly been produced as a process product using renewable energy, preferably at a geographical location with good availability of renewable energy, in order subsequently to provide this methanol for the process of the invention either by delivery by transport in containers or by a methanol stream in a continuous process. For provision in the context of a continuous process, methanol production is preferably in fluid connection with production of the hexamethylenediamine, for example via a pipe conduit.

Methanol production facilities are licensable on the market, for example from Air Liquide, Johnson Matthey and others. An overview of the classical production of methanol from synthesis gas is published for example in Ott et al., Methanol in: Ullmann's Encyclopedia of industrial chemistry, 2012, Wiley-VCH Verlag, Weinheim (doi 10.1002/14356007.a16_465.pub3), which is fully and expressly incorporated by reference.

The methanol provided as described previously according to step a) i) is converted into propene in step a) ii) of the process according to the invention for providing the hexamethylenediamine by a process containing at least the following steps:
provision of propene as a product of a process comprising at least the following steps:
ii-1) conversion of the previously provided methanol to afford a product mixture containing dimethyl ether, water and methanol;
ii-2) conversion of starting material containing dimethyl ether, water and methanol in each case from the aforementioned product mixture at a temperature of more than 200° C. by contact with a catalyst, preferably with at least one zeolite compound, into propene.

Steps ii-1) and ii-2) are part of the methanol-to-propylene process (also known as the MTP process). The MTP process was first developed by Lurgi.

In an MTP process, gaseous methanol for example is reacted in a reactor over a catalyst to afford a product mixture containing dimethyl ether, water and methanol in the sense of step ii-1) of the process according to the invention. Suitable reactors include for example a fixed-bed reactor or a fluidized-bed reactor.

In a preferred embodiment of the process according to the invention, the reaction of the provided methanol according to step a) ii-1) is carried out by contacting the methanol, preferably the methanol in the gas phase, with a catalyst.

An example of a suitable particularly preferred catalyst for converting the methanol in step ii-1) is Al₂O₃ pellets as described in EP 0 448 000 B1 or DE 197 23 363 A1.

Before the conversion of the methanol in step a) ii-1), the provided methanol is preferably brought to a temperature in a range from 200° C. to 350° C. and the conversion is performed with this correspondingly temperature-controlled methanol. After the conversion, the product mixture containing dimethyl ether, methanol and water in turn preferably has a temperature of 350° C. to 450° C.

The product mixture from step ii-1), especially in the form of a gas stream, is, optionally after performing optional purification steps, introduced for example into a further reactor as the starting material for step ii-2) and therein converted into propene over a catalyst. An example of a suitable catalyst is the catalyst marketed by Clariant under the trade name MTPROP® or a zeolite compound catalyst, such as is described for example in U.S. Pat. No. 7,015,369 B2, column 1, lines 43-52, and Examples 1 and 2. In a further embodiment of the process according to the invention, the starting material is preferably converted into propene by contact with a zeolite compound as catalyst.

It is preferable when in step ii-2) the starting material is reacted at a temperature of 350° C. to 600° C., especially at a temperature of 380° C. to 550° C.

The propene provided by step ii-2) of the process according to the invention is reacted with ammonia to afford acrylonitrile, optionally after separation of secondary constituents, according to step iii). For the provision of the hexamethylenediamine it is sufficient according to the invention if the acrylonitrile employed therefor is an actual process product of at least the steps i), ii) and iii) recited under a). This means that it is sufficient for performing the step of provision of the hexamethylenediamine merely to withdraw the acrylonitrile produced in the recited manner from a storage vessel or from a feed conduit in the context of a delivery as raw material in order to supply it at least to step a) iv) of the process according to the invention. In this case the hexamethylene diisocyanate producer, as the performer of the process according to the invention, does not themself perform the steps i), ii) and iii) for producing the acrylonitrile recited under a) of the process according to the invention but rather only ensures that the provided hexamethylenediamine was produced from acrylonitrile that was synthesized by application of at least the steps i), ii) and iii) recited under a) and is thus the actual process product thereof.

For the synthesis of acrylonitrile from the propene ammonia is initially provided as the actual process product of a process comprising at least the following steps:
iii-1-1) provision of hydrogen gas by electrolysis of water, preferably using electrical energy generated from renewable energy;
iii-1-2) reaction of the provided hydrogen gas with gaseous nitrogen to afford ammonia.

The electrolysis of water to be carried out in step iii-1-1) for synthesis of the hydrogen gas to be provided may be carried out with plants according to the prior art. Industrial systems for alkaline water electrolysis as well as for polymer electrolyte-based electrolysis, so-called PEM electrolysis, are known and commercially available. The principles of water electrolysis are described by way of example in chapter 6.3.4 in Volkmar M. Schmidt in “Elektrochemische Verfahrenstechnik” [Electrochemical process technology](2003 Wiley-VCH-Verlag; ISBN 3-527-29958-0).

The production of the ammonia is carried out according to step a) iii-1-2) starting from nitrogen and the hydrogen provided in the preceding step a) iii-1-1) in a manner very familiar to a person skilled in the art, for example as described by the Haber-Bosch process using known reactor technologies and designs for this process which are fully and expressly incorporated here by reference.

For the provision/the production of the ammonia it has proven advantageous when the nitrogen used therefor is provided by an air separation. Processes for fractionation of air and correspondingly suitable plants for ammonia production are part of the prior art and are available on the market.

The propene provided by at least the aforementioned process steps is then oxidatively reacted with the provided ammonia to afford acrylonitrile, preferably according to the so-called SOHIO process (for example according to U.S. Pat. No. 2,904,580 A). It is preferable according to the invention when the reaction of the provided ammonia with the propene according to step a) iii-2) is carried out using oxygen from air at a temperature of 350° C. to 550° C.

In a further embodiment of the process for producing the provided acrylonitrile the reaction is particularly preferably carried out at a relative pressure of 40 to 220 kPa.

It has proven advantageous to carry out the reaction of the provided ammonia with the propene according to step a) iii-2) over a molybdate- and/or antimonate-based catalyst. In this embodiment it is in turn preferable when the aforementioned temperature is established and the reaction is carried out using oxygen, preferably oxygen from air.

The acrylonitrile produced according to step iii-2) of the process according to the invention is electrochemically converted into adiponitrile (CAS No. 111-69-3, also known as adipic acid dinitrile) according to step iv). For provision of the hexamethylenediamine it is sufficient according to the invention when the adiponitrile employed therefor is an actual process product of at least the steps i), ii), iii) and iv) recited under a). This means that it is sufficient for performing the step of provision of the hexamethylenediamine merely to withdraw the adiponitrile produced in the recited manner from a storage vessel or from a feed conduit in the context of a delivery as raw material in order to supply it at least to step a) v) of the process according to the invention. In this case the hexamethylene diisocyanate producer, as the performer of the process according to the invention, does not themself perform the steps i), ii) and iii) and iv) for producing the adiponitrile recited under a) of the process according to the invention but rather only ensures that the provided hexamethylenediamine was produced from adiponitrile that was synthesized by application of at least the steps i), ii), iii) and iv) recited under a) and is thus the actual process product thereof.

Said adiponitrile must be an actual process product of at least the steps
introduction of a mixture containing water, said acrylonitrile from step a) iii-2) and at least one electrolyte salt into the cathode space of an electrolysis cell and contacting the mixture with a cathode having an electrical current applied to it;
cathodic hydrodimerization of acrylonitrile to form an adiponitrile-containing product mixture;
discharging of the adiponitrile-containing product mixture from the cathode space of the electrolysis cell and optional purification of the adiponitrile.

These steps are carried out for example as described in U.S. Pat. No. 3,193,480 A which is fully and expressly incorporated here by reference.

To provide the mixture at least water, said acrylonitrile and at least one electrolyte salt are mixed. This preferably forms an emulsion of acrylonitrile-containing droplets in water.

In a preferred embodiment a suitable electrolyte salt in the cathode space of the electrolysis cell is at least one quaternary ammonium salt, especially at least one quaternary ammonium sulfonate. Particularly preferred electrolyte salts are selected from at least one electrolyte salt from the group formed from tetraalkylammonium salts, tetraalkanolammonium salts, dialkyldialkanolammonium salts, alkyltrialkanolammonium salts, trialkylalkanolammonium salts, trialkylbenzylammonium salts, quaternary N-heterocyclo-N-alkylammonium salts, wherein the sulfonic acid salts thereof are in turn more preferably suitable. Very particularly preferred alkyl groups of the ammonium cations of the aforementioned salts include (C₁-C₆)-alkyl groups such as methyl, ethyl, n-propyl, isopropyl, n-butyl, sec-butyl, isobutyl, tert-butyl. Very particularly preferred alkanol groups include (C₁-C₆)-hydroxyalkyl groups such as 2-hydroxyethyl, 2-hydroxypropyl, 3-hydroxypropyl, 4-hydroxypropyl.

Particularly preferably suitable N-heterocyclo groups include for example piperidinium, morpholinium and pyrrolidinium.

Preferably suitable anions of the quaternary ammonium salts include chloride, fluoride, sulfates, phosphates and sulfonates.

The tetraalkylammonium salts of aryl- or alkylarylsulfonic acids particularly preferably include electrolyte salts. Particularly preferred sulfonate anions of the quaternary ammonium salts preferably usable as an electrolyte salt include benzenesulfonate, o-tolylsulfonate, m-tolylsulfonate, o-tolylsulfonate, m-tolylsulfonate, p-tolylsulfonate, o-cumylsulfonate, m-cumylsulfonate, p-cumylsulfonate, alpha-naphthylsulfonate, beta-naphthylsulfonate, p-xylylsulfonate or mixtures thereof. Particularly preferred sulfate anions are selected from at least one anion having an organic radical analogous to the organic radicals of the aforementioned sulfonate anions.

In a most preferred embodiment at least one electrolyte salt is selected from tetramethylammonium chloride, tetraethylammonium chloride, tetra(n-butyl)ammonium chloride, tetraethylammonium p-tolylsulfonate, tetraethylammonium o-tolylsulfonate, tetraethylammonium m-tolylsulfonate, tetraethylammonium benzenesulfonate; tetraethylammonium o-, m- or p-cumylsulfonate or o-, in-, or p-ethylbenzenesulfonate; N, N-dimethylpiperidinium o-, m- or p-tolylsulfonate or o-, m- or p-biphenylsulfonate; tetrabutylammonium alpha- or beta-naphthylsulfonate or o-, m- or p-tolylsulfonate; tetrapropylammonium o-, m- or p-amylbenzenesulfonate or alpha-ethyl-beta-naphthylsulfonate; tetra(2-hydroxylethyl)ammonium o-, m- or p-cumylsulfonate or o-, m- or p-tolylsulfonate; tetra(4-hydroxybutyl)ammonium benzenesulfonate or p-xylyl-3-sulfonate; tetrapentylammonium o-, m- or p-tolylsulfonate or o-, m- or p-hexylbenzenesulfonate, tetrapentanolammonium p-cumyl-3-sulfonate or benzenesulfonate; methyltriethylammonium o-, m- or p-tolylsulfonate or mesityl-2-sulfonate; trimethylethylammonium o-xylyl-4-sulfonate or o-, m- or p-tolylsulfonate; triethylpentylammonium alpha- or beta-naphthylsulfonate or o-, m- or p-butylbenzenesulfonate, trimethylhydroxyethylammonium benzenesulfonate or o-, m- or p-tolylsulfonate; N, N-diethylpiperidinium or N-methylpyrrolidinium o-, m- or p-hexylbenzenesulfonate or o-, m- or p-tolylsulfonate, N, N-diisopropyl or N, N-dibutylmorpholinium o-, m- or p-tolylsulfonate or o-, m- or p-biphenylsulfonate or mixtures of the aforementioned salts.

In a further preferred embodiment it is advantageous when the mixture introduced in step a) iv) has a pH in the range from pH 7 to pH 10, in particular from pH 7 to pH 9.5. It is further particularly preferable when the pH of the mixture in the cathode space is controlled via the normality of the anolyte in the anode space of the electrolysis cell and the formal migration of H⁺ ions from the anode space into the cathode space. To this end the anolyte in the anode chamber of the electrolysis cell preferably has a normality in the range from 0.4 to 1.2.

In a preferred embodiment the electrolysis cell in which the electrochemical hydrodimerization is carried out comprises a cathode space and an anode space separated from one another by a membrane. Membranes suitable therefor include for example the membranes mentioned in U.S. Pat. No. 3,193,480 A or are commercially available, for example under the trade name Nafion® 115.

In a further embodiment of the process it is preferable when the electrolysis cell has an electrical current having a current density of 15 to 40 A/dm², particularly preferably at a voltage of 5 to 20 volts, applied to it.

According to the invention it is further preferable when the cathodic hydrodimerization of the acrylonitrile to adiponitrile is performed via an applied electrical current which was in fact generated using renewable energy, especially selected from wind power, hydropower, solar power or mixtures thereof.

The adiponitrile provided in step a) iv) is converted into hexamethylenediamine via a catalytic hydrogenation with hydrogen by processes known to those skilled in the art (preferably at a temperature in the range from 100° C. to 200° C. and an operating pressure in the range from 28 to 41 MPa). The hydrogenation is more preferably performed in the liquid phase, wherein ammonia is optionally added as a medium for heat transfer. Catalysts suitable for catalytic hydrogenation of adiponitrile especially include cobalt-containing catalysts, such as are described for example in U.S. Pat. Nos. 3,232,888 A, 3,821,305 A, 3,773,832 or 4,598,058 A. Likewise usable are iron-containing catalysts, such as are described for example in U.S. Pat. No. 3,696,153 A or U.S. Pat. No. 4,587,228 A.

Step b) of the process according to the invention comprises producing phosgene. This comprises initially providing in step b) i) of the process according to the invention carbon monoxide which is an actual process product of at least the methods recited in step b) i).

It is sufficient for the provision of the carbon monoxide in the context of the invention when the carbon monoxide is a process product of at least the methods recited in step b) i). This means that, when performing the process according to the invention, it is sufficient for the execution of the step of providing the carbon monoxide even to merely draw said carbon monoxide as an actual process product of said steps from a storage vessel or from a feed conduit in the context of a delivery as raw material. In this case the hexamethylene diisocyanate producer, as the performer of the process according to the invention, does not themself perform the production of the carbon monoxide, but rather only ensures that the provided carbon monoxide has been correspondingly produced by a supplier and is an actual process product at least of the methods recited in step b) i).

In one embodiment it is likewise possible according to the invention when to provide the carbon monoxide the aforementioned steps for carbon monoxide production are performed by the hexamethylene diisocyanate producer themself in step b) i) as integral steps of a process according to the invention and the resulting carbon monoxide is sent to the phosgene production in step b) ii).

The embodiments of the partial reduction of CO₂ to CO recited in step b) i) and the partial oxidation of organic material to CO, wherein at least one organic material is selected from at least one organic solid compound, methane from a biological source or mixtures thereof, have already been described under step a) i-1) (vide supra). These also apply correspondingly to step b) i).

In step b) i) the carbon monoxide may also derive from a partial oxidation of at least one monohydroxyalkyl compound, especially of methanol. This step is referred to as catalytic decomposition hereinbelow. It is preferable when the monohydroxylalkyl compound is methanol and the methanol is a process product of at least the steps described under a) i). The embodiments described under step a) i) are again to be taken into account (vide supra).

In the catalytic decomposition step of the methanol, methanol provided is in a preferred embodiment catalytically decomposed by contacting of gaseous methanol with a catalyst to form carbon monoxide and optionally also hydrogen gas. If the methanol is not in gaseous form for this purpose (for example in the case of withdrawal from a storage vessel), it is subjected to evaporation beforehand. The energy input needed for the evaporation can be generated by utilization of fossil fuels, for example natural gas, as an energy source. To establish the temperature necessary for evaporation the process according to the invention is supplied with thermal energy which is provided by at least one method selected from (i) combustion of fuel containing hydrogen (gaseous H₂) produced using renewable energy, (ii) combustion of fuel containing methane from a biological source or (iii) conversion of electrical energy generated from renewable energy into heat. It is particularly preferable here in turn when the renewable energy used is either wind power, solar energy, hydro power or mixtures thereof.

It is preferable when gaseous methanol is passed over a catalyst whose active component comprises at least one transition metal species for catalytic decomposition. A transition metal species is understood by a person skilled in the art to mean all d-block elements and chemical compounds thereof. The catalyst is particularly preferably a transition metal species applied to a support. Very particular preference is given to at least one transition metal species selected from group VII, VIII or XI of the Periodic Table of the Elements, preferably applied in turn to a support.

The catalytic decomposition of methanol is preferably performed at a temperature below 500° C., more preferably below 400° C. If an energy input is necessary for the catalytic decomposition of methanol this may be brought about by utilization of fossil fuels, for example natural gas, as an energy source. To establish the temperature necessary for catalytic decomposition of the methanol, the process according to the invention is particularly preferably supplied with thermal energy which is provided by at least one method selected from (i) combustion of fuel containing hydrogen (gaseous H₂) produced using renewable energy, (ii) combustion of fuel containing methane from a biological source or (iii) conversion of electrical energy generated from renewable energy into heat. It is particularly preferable here in turn when the renewable energy used is either wind power, solar energy, hydro power or mixtures thereof.

The catalytic decomposition of methanol is preferably performed at a temperature of at least 200° C. It is in turn preferable when the catalytic decomposition of the methanol is carried out at a temperature of 200° C. to below 500° C., particularly preferably of 200° C. to below 400° C.

The catalytic decomposition of methanol is preferably performed at an absolute pressure of below 50 bar, more preferably of below 40 bar.

The product gas obtained from the catalytic decomposition contains not only carbon monoxide but often also methanol, carbon dioxide, hydrogen and water and optionally byproducts that may derive inter alia from the group of ethers (dimethyl ether), alcohols (ethanol), aldehydes or esters. Prior to the reaction with chlorine to afford phosgene the obtained product gas from the catalytic decomposition is preferably subjected, in a preferred, additional process step, to a purification step in which methanol, carbon dioxide, hydrogen and water and optionally byproducts of carbon monoxide are separated.

The carbon monoxide provided for step b) ii) is preferably freed of secondary constituents and purified before phosgene production. To this end the carbon monoxide is preferably sent for carbon dioxide separation in a CO₂ separation unit in which the CO₂ is separated.

The CO₂ separation unit and thus the separation of CO₂ may be in the form of an “amine scrubbing”, wherein the carbon monoxide-containing product gas from the reformer process is especially subjected to a scrubbing of the gas mixture by the principle of chemisorption with amines, such as monoethanolamine (MEA), diethanolamine (DEA), methyldiethanolamine (MDEA) or diglycolamine (DGA), as is known in principle and achieves a high purity of the purified gas mixture in an absorption column.

In one embodiment of the process which is further preferable according to the invention a separation of water from the provided carbon monoxide is carried out.

In a further preferred embodiment of the process the separation of carbon dioxide is carried out once water has been previously separated from the provided carbon monoxide. To this end the provided carbon monoxide is initially sent to a water separation unit, the water is separated therein and the carbon monoxide-containing, dry product gas obtained after the separation of water is sent to a CO₂ separation unit in which the CO₂ is separated. In the water separation unit, separation of the water is effected for example by cooling the provided carbon monoxide and separating the water as condensate for example.

A variant of the process which is preferable according to the invention is a process in which the provided carbon monoxide (preferably previously freed of water and of CO₂) is introduced into an H₂—CO separation unit in which hydrogen is separated. This forms at least one gas stream, wherein the gas at 25° C. and 1013 mbar contains at least 95% by volume of carbon monoxide, more preferably at least 99% by volume of carbon monoxide. Said introduced carbon monoxide is preferably initially separated into two gas streams in the H₂—CO separation unit. This forms a gas in the form of a gas stream containing at least 95% by volume (preferably at least 99% by weight) of carbon monoxide, a further gas in the form of a gas stream whose greatest constituent is hydrogen and inter alia contains carbon monoxide. The further gas is also referred to as tail gas from the H₂—CO separation or, if there is no tail gas treatment, as end gas. An H₂—CO separation unit that works by this principle of separation is the so-called cold box. The hydrogen-containing residual gas from the H₂—CO separation may be reintroduced into the production of methanol.

The carbon monoxide obtained (optionally after purification) is reacted with chlorine to afford phosgene in a next process step b) ii), preferably over a catalyst. More preferably, the catalyst is activated carbon.

For the provision of the chlorine for the synthesis of phosgene effective in this embodiment, those skilled in the art will be well acquainted with the production of chlorine gas from electrochemical oxidation by the electrolysis of hydrochloric acid with a gas-diffusion electrode (also referred to as the HCl ODC electrolysis process (ODC=oxygen-depleting electrode)); for a suitable electrolysis cell see U.S. Pat. No. 6,022,634 A, WO 03/31690 A1), the production of chlorine gas from hydrochloric acid diaphragm electrolysis (see EP 1 103 636 A1), the production of chlorine gas from thermocatalytic gas-phase oxidation (see WO 2012/025483 A2), and the production of chlorine from chloralkali electrolysis (see WO 2009/007366 A2). Reference is made expressly and in full to the content of the abovementioned documents cited in connection with the production of chlorine gas. The chlorine required for the synthesis of phosgene is in a preferred variant of this embodiment of the process produced electrolytically, especially through electrochemical oxidation by hydrochloric acid electrolysis with a gas-diffusion electrode, through electrochemical oxidation by hydrochloric acid diaphragm electrolysis or through electrochemical oxidation by chloralkali electrolysis. It is in this case in turn particularly preferable when said electrochemical oxidation is in each case carried out using electricity generated from renewable energy, especially from renewable energy in the form of wind power, solar energy or hydro power.

In step c) of the process according to the invention phosgene is reacted with the hexamethylenediamine provided according to step a) and the hexamethylene diisocyanate is produced. Corresponding processes and apparatuses for reaction of phosgene with organic amine compounds are known to those skilled in the art, for example from WO 2017/093215 A1 which is fully and expressly incorporated here by reference. It has proven advantageous in the context of the present process when in a preferred embodiment the phosgene and the hexamethylenediamine are reacted in the gas phase to obtain hexamethylene diisocyanate. The phosgenation of amines in the gas phase is known per se and may be carried out for example as described in EP 0 289 840 B1, EP 1 319 655 A2, EP 1 555 258 A1, EP 1 275 639, A1, EP 1 275 640 A1, EP 1 449 826 A1, EP 1 754 698 B1, DE 10 359 627 A1, DE 10 2005 042392 A1 or WO 2017/093215 A1.

A second subject of the invention is a process for producing polyurethane comprising at least the following steps:
provision of hexamethylene diisocyanate which is a process product of the process of the first subject of the invention;
provision of at least one organic polyol;
reaction of said hexamethylene diisocyanate with said organic polyol to afford polyurethane.

Organic polyols are to be understood as meaning organic compounds having at least two hydroxy groups. These are preferably selected from polyester polyol, polyether polyol, polycarbonate polyol, polyether ester polyol, polyacrylate polyol, polyester polyacrylate polyol or mixtures thereof, particularly preferably selected from the group of polyether polyols and/or polyester polyols.

In a preferred embodiment the process for producing polyurethane is characterized in that the provision of the hexamethylene diisocyanate is carried out using a multi-component system according to the third subject of the invention (vide infra).

A third subject of the invention is a multi-component system for hexamethylene diisocyanate production according to a production process of the first subject of the invention, comprising
i) a volume filled with hexamethylenediamine which is a process product of step a) of the process according to the first subject of the invention;
ii) a volume filled with carbon monoxide which is a process product of step b) i-1) of the process according to the first subject of the invention;
iii) at least one electrolysis apparatus for production of chlorine;
iv) at least one reactor for production of phosgene, wherein the reactor has at least one inlet for chlorine which is in fluid connection with the electrolysis apparatus for production of chlorine and at least one inlet for carbon monoxide which is in fluid connection with the volume filled with carbon monoxide;
v) at least one reactor for production of hexamethylene diisocyanate comprising at least one inlet for said phosgene and at least one inlet for said hexamethylenediamine, wherein the inlet for said phosgene is in fluid connection with the reactor for production of phosgene and the inlet for said hexamethylenediamine is in fluid connection with the volume of said hexamethylenediamine.

All embodiments of step a) of the process according to the invention for producing hexamethylene diisocyanate also apply mutatis mutandis to the multicomponent system.

All embodiments of step b) of the process according to the invention for producing hexamethylene diisocyanate also apply mutatis mutandis to the multicomponent system.

A fourth subject of the invention is therefore the use of methanol as raw material for producing the hexamethylene radical of hexamethylene diisocyanate. Preference is given to a corresponding use which is carried out by a process according to the first subject of the invention.

1. A process for producing hexamethylene diisocyanate for the production of polyurethane, the process comprising:
a) provision of hexamethylenediamine which is a process product of a process comprising at least the following steps:
i) provision of methanol which is a process product of a process comprising at least the following steps:
i-1) provision of carbon monoxide as a process product of at least a partial reduction of CO₂ to CO and/or as a process product of at least a partial oxidation of organic material to CO, wherein at least one organic material is selected from the group consisting of at least one organic, solid compound, methane from a biological source, and mixtures thereof; and
i-2) conversion of the provided carbon monoxide into methanol;
ii) synthesis of propene from the methanol by at least the following steps:
ii-1) conversion of the methanol to afford a product mixture containing dimethyl ether, water and methanol; and
ii-2) conversion of a starting material containing dimethyl ether, water and methanol in each case from the product mixture at a temperature of more than 200° C. by contact with a catalyst into propene;
(iii) synthesis of acrylonitrile from the propene by at least the following steps:
iii-1) provision of ammonia which is a process product of a process comprising at least the following steps:
iii-1-1) provision of hydrogen gas as a product of an electrolysis of water; and
iii-1-2) reaction of the hydrogen gas with gaseous nitrogen to afford ammonia; and
iii-2) reaction of the propene with the ammonia to afford acrylonitrile;
iv) synthesis of adiponitrile from the acrylonitrile by at least the following steps:
introduction of a mixture containing water, said acrylonitrile and at least one electrolyte salt into a cathode space of an electrolysis cell and contacting the mixture with a cathode having an electrical current applied to it;
cathodic hydrodimerization of acrylonitrile to form an adiponitrile-containing product mixture; and
discharging of the adiponitrile-containing product mixture from the cathode space of the electrolysis cell; and
v) synthesis of hexamethylenediamine from the acrylonitrile by at least the following steps:
provision of hydrogen gas as a product of an electrolysis; and
hydrogenation of the adiponitrile with the hydrogen gas to afford hexamethylenediamine;
b) production of phosgene by at least the following process steps:
i) provision of carbon monoxide as a process product of at least a partial reduction of CO₂ to CO and/or as a process product of at least a partial oxidation of organic material to CO, wherein at least one organic material is selected from the group consisting of at least one organic, solid compound, an organic, liquid monohydroxyalkyl compound, methane from a biological source, and mixtures thereof;
ii) reaction of the carbon monoxide from step i) with chlorine to afford phosgene; and
c) reaction of the hexamethylenediamine with the phosgene to afford hexamethylene diisocyanate.

2. The process as claimed in claim 1, wherein the carbon monoxide provided in step a) i-1) and/or the carbon monoxide provided in step b) i-1) is in each case independently a process product of at least a partial reduction of a gas stream comprising at least COₓ where x=1 or 2.

3. The process as claimed in claim 1, wherein the carbon monoxide provided in step a) i-1) and/or the carbon monoxide provided in step b) i-1) is in each case independently a process product of at least a partial reduction of CO₂ to CO.

4. The process as claimed in claim 1, wherein the carbon monoxide provided in step a) i-1) and/or the carbon monoxide provided in step b) i-1) is in each case independently a process product of at least a reverse water gas shift reaction using H₂ provided by electrolysis or is provided from an electrochemical reduction of CO₂ to CO or from mixtures thereof with the proviso that electrical energy produced from renewable energy is in each case utilized here.

5. The process as claimed in claim 1, wherein the conversion of the methanol according to step a) ii-1) is carried out by contacting the methanol, with a catalyst.

6. The process as claimed in claim 1, wherein for the conversion of the methanol in step a) ii-1), said methanol has a temperature in a range from 200° C. to 350° C.

7. The process as claimed in claim 1, wherein the conversion of the starting material according to step a) ii-2) is carried out at a temperature of 350° C.

8. The process as claimed in claim 1, wherein the reaction of the ammonia with the propene according to step a) iii-2) is carried out using oxygen from air at a temperature of 350° C. to 550° C.

9. The process as claimed in claim 1, wherein the reaction of the ammonia with the propene according to step a) iii-2) is carried out over a molybdate- and/or antimonate-based catalyst.

10. The process as claimed in claim 1, wherein the electrolyte salt in the cathode space of the electrolysis cell is selected from the group consisting of at least one quaternary ammonium salt.

11. The process as claimed in claim 1, wherein in step a) iv) the electrolysis cell comprises the cathode space and an anode space separated from one another by a membrane.

12. The process as claimed in claim 1, wherein in step a) iv) the introduced mixture has a pH in a range from pH 7 to pH 10.

13. The process as claimed in claim 1, wherein the electrolysis cell has an electrical current having a current density of 15 to 40 A/dm² applied to it.

14. A process for producing polyurethane, the process comprising:
production of hexamethylene diisocyanate which is a process product of the process as claimed in claim 1;
provision of at least one organic polyol; and
reaction of said hexamethylene diisocyanate with said polyol to afford polyurethane.

15. The process as claimed in claim 14, wherein the provision of the hexamethylene diisocyanate is carried out using a multicomponent system for hexamethylene diisocyanate production, the multicomponent system comprising:
i) a volume filled with hexamethylenediamine, which is a process product of step a) of the process;
ii) a volume filled with carbon monoxide which is a process product of step b) i-1) of the process;
iii) at least one electrolysis apparatus for production of chlorine;
iv) at least one reactor for production of phosgene, wherein the reactor has at least one inlet for chlorine which is in fluid connection with the electrolysis apparatus for production of chlorine and at least one inlet for carbon monoxide which is in fluid connection with the volume filled with carbon monoxide; and
v) at least one reactor for production of hexamethylene diisocyanate comprising at least one inlet for said phosgene and at least one inlet for said hexamethylenediamine, wherein the inlet for said phosgene is in fluid connection with the reactor for production of phosgene and the inlet for said hexamethylenediamine is in fluid connection with the volume of said hexamethylenediamine.

16. A multicomponent system for hexamethylene diisocyanate production by a production process as claimed in claim 1, the multicomponent system comprising:
i) a volume filled with hexamethylenediamine, which is a process product of step a) of the process;
ii) a volume filled with carbon monoxide which is a process product of step b) i-1) of the process;
iii) at least one electrolysis apparatus for production of chlorine;
iv) at least one reactor for production of phosgene, wherein the reactor has at least one inlet for chlorine which is in fluid connection with the electrolysis apparatus for production of chlorine and at least one inlet for carbon monoxide which is in fluid connection with the volume filled with carbon monoxide; and
v) at least one reactor for production of hexamethylene diisocyanate comprising at least one inlet for said phosgene and at least one inlet for said hexamethylenediamine, wherein the inlet for said phosgene is in fluid connection with the reactor for production of phosgene and the inlet for said hexamethylenediamine is in fluid connection with the volume of said hexamethylenediamine.

17. The process as claimed in claim 1 comprising utilizing methanol as raw material for producing a hexamethylene radical of hexamethylene diisocyanate.

18. The process as claimed in claim 1, wherein:
a) the catalyst comprises at at least one zeolite compound; and/or
b) the process further comprises purification of the adiponitrile.

19. The process as claimed in claim 2, wherein the gas stream further comprises hydrogen gas.

20. The process as claimed in claim 5, wherein the methanol is contacted in the gas phase.
